SEQUENCE LISTING (4 sequences)

SEQ ID NO: 1 - DNA, 642 bp, Artificial
  FABP
gggagaccacaacggtttccctctagaaataattttgtttaactttaagaaggagataaacaatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaagtggaatggacaagagacatcacttgtgcgggagatggttgacgggaaactcattctgacactcacccacggcactgcagtttgcactcgtacttacgagaaacaggcataataactaactaaccaagatctgtaccccttggggcctctaaacgggtcttgaggggttttttgcccgggatccggctgctaacaaagcccgaaaggaagctgagttggctgctgccaccgctgagcaataactagcataaccccttggggcctctaaacgggtcttgaggggttttttg

SEQ ID NO: 2 - DNA, 3701 bp, Artificial
  plasmid pXFA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctatttaggtgacactatagaatacatcgattaatacgactcactatagggagaccacaacggtttccctctagaaataattttgtttaactttaagaaggagataaacaatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaagtggaatggacaagagacatcacttgtgcgggagatggttgacgggaaactcattctgacactcacccacggcactgcagtttgcactcgtacttacgagaaacaggcataataactaactaaccaagatctgtaccccttggggcctctaaacgggtcttgaggggttttttgcccgggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 3688 bp, Artificial
  plasmid pIX4.0-FA-A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcctaatacgactcactatagggtctagataagtattttactgttttcgtaacagttttgtaataaaaaaacctataaatccatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaagtggaatggacaagagacatcacttgtgcgggagatggttgacgggaaactcattctgacactcacccacggcactgcagtttgcactcgtacttacgagaaacaggcatgacctgccctctcctcccactgactgctcttctgtcagtgggctactcctggactcggcaccagattgcctcatttttctcctctggcattttgtataaatccaccttgactggggaaattctcctggggtcaggtggcaccagcctggatctaaataataagtaattaactagttaagtaaccaagatccgtaagctagcacaaccgcgtctcaaaaaaaaaaaaaaaaaaaaaaaaaaaaaaaaaaaaaaaaaaaaaaaaaaaaaaaaaaaaaaacagtcttcgctgagcaataactagcataaccccttggggcctctaaacgggtcttgaggggttttttgatatcccgggatttaaataggcctgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 739 bp, Artificial
  transkription product of plasmid pIX4.0-FA-A
gggtctagataagtattttactgttttcgtaacagttttgtaataaaaaaacctataaatccatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaagtggaatggacaagagacatcacttgtgcgggagatggttgacgggaaactcattctgacactcacccacggcactgcagtttgcactcgtacttacgagaaacaggcatgacctgccctctcctcccactgactgctcttctgtcagtgggctactcctggactcggcaccagattgcctcatttttctcctctggcattttgtataaatccaccttgactggggaaattctcctggggtcaggtggcaccagcctggatctaaataataagtaattaactagttaagtaaccaagatccgtaagctagcacaaccgcgtctcaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2008/0096256 A1
(43) Pub. Date: Apr. 24, 2008

(54) Method for gene expression

(76) Inventors: Helmut Merk, Berlin (DE); Wolfgang Stiege, Berlin (DE); Stefan Kubick, Berlin (DE)

Correspondence Address: MAYER & WILLIAMS PC, 251 NORTH AVENUE WEST, 2ND FLOOR, WESTFIELD, NJ, 07090, US

(21) Appl. No.: 11/731,445

(22) Filed: Mar. 30, 2007

(30) Foreign Application Priority Data
Mar. 30, 2006 (DE) 10 2006 015 321.9

(51) Int. Cl. C07H 21/04; C12P 19/34

(52) U.S. Cl. 435/91.2; 536/23.1

(57) ABSTRACT

The invention relates to a method for gene expression in a cell-free translation system, wherein the reaction solution comprises an RNA matrix with a gene sequence, which codes for an expression product to be expressed, and a translation system from eukaryotic cells, wherein the reaction solution is incubated, and wherein the expression product is optionally separated from the reaction solution, characterized by that the RNA matrix, viewed in the 5′-3′ direction, comprises a Shine Dalgarno sequence, connected to a first spacer sequence and connected to the gene sequence.

### FIELD OF THE INVENTION

The invention relates to a method for gene expression in a cell-free translation system, wherein the reaction solution comprises an RNA matrix with a gene sequence, which codes for an expression product to be expressed, and a translation system from eukaryotic cells, wherein the reaction solution is incubated, and wherein the expression product optionally is separated from the reaction solution. The invention furthermore relates to a kit for carrying-out such a method and an RNA to be used in such a method.

### PRIOR ART AND BACKGROUND OF THE INVENTION

Methods for cell-free expression of proteins are, for instance, known from the documents EP 0312 617 B1, EP 0401 369 B1 and EP 0 593 757 B1.

Accordingly, the components necessary for transcription and/or translation are incubated, in addition to a nucleic acid strand coding for a desired protein, in a reaction vessel, and after expression the polypeptides/proteins are isolated from the reaction solution.

The components necessary for transcription as well as those necessary for translation can easily be obtained from the supernatants of prokaryotic or eukaryotic cell lysates after for instance, a 30,000×g centrifugation. This so-called “S-30 extract” contains all components necessary for transcription and translation.

The expression typically takes place at 27° C. or 37° C., but may, however, also take place at temperatures from 17° C. to 45° C. The adjustment of the temperature is, in particular, recommended for the expression of proteins, in which a complicated secondary/tertiary structure is to be formed. By lowering the temperature, the synthesis rate can be lowered and thus the proteins are given the possibility to correctly fold, in order that a functioning/active protein is obtained.

In the method for cell-free expression of proteins disclosed in the document EP 0312 617 B1, the nucleic acid strand coding for the protein is added to the reaction solution as mRNA. Thereby, for the production of polypeptides in the cell-free system, only the components of the translation apparatus necessary for translation, in particular ribosomes, initiation, elongation, release factors and aminoacyl-tRNA-synthetase, as well as amino acids and ATP and GTP as energy-supplying substances, need to be placed into a reaction vessel. In the following polypeptide/protein synthesis, besides polypeptides/proteins, also low-molecular substances, such as ADP, AMP, GDP, GMP and inorganic phosphates are formed, under consumption of the energy supplying substances ATP and GTP and of amino acids. For maintaining the reaction, the substances consumed during translation can be guided out during the translation, and at the same time the energy supplying substances and the amino acids can be guided in for maintaining the initial concentration.

The document EP 0401 369 B1 discloses a method, wherein the nucleic acid coding for the protein can be added as mRNA or DNA to the reaction solution. The latter has the advantage that DNA is substantially more stable than mRNA, and the necessary (if applicable separately performed) transcription process of the DNA into RNA is not necessary before the reaction, but the DNA can directly be used, for instance, as a vector or a linear construct. By using the DNA, the cell-free expression system must further contain, besides the translation factors mentioned above, the transcription factors necessary for the transcription of the DNA into RNA, such as, for instance, RNA polymerase, factor and rho-protein and the nucleotides ATP, UTP, GTP and CTP. Here, too, the low-molecular substances consumed during the transcription/translation, such as ADP, AMP, GDP, GMP and inorganic phosphates can be guided out during the translation and at the same time the energy supplying substances, nucleotides and the amino acids can be guided in for maintaining the initial concentration.

Further methods for gene expression are for instance known from the documents, DE 101 37 792 A and DE 10 2004 032 460 A. A method for the production of a lysate for the gene expression is known from the document DE 103 36 705 A.

The Shine Dalgarno sequence (Shine, J., Dalgarno, L., Nature, 254(5495):34-38 (1975) is a partial sequence in the mRNA of prokaryotes, which is detected by the ribosomes and thus marks the starting point of the translation. It is typically at the 5′ side of the first coding AUG and mainly consists of purines. The sequence is typical for the species and may be taken for various species from the publicly accessible gene data banks. In contrast thereto, the RNA of eukaryotes typically comprises, for the initialization sequence, the Kozak sequence. In the insect cell system, the Kozak sequence has, however, only a very small role. Herein, the translation efficiency is considerably increased by means of the 5′ UTR of the polyhedrin gene from the Baculovirus Autographa californica (Raming K, Krieger J, Strotmann J, Boekhoff I, Kubick S, Baumstark C and Breer H, 1993, Cloning and expression of odorant receptors, Nature 361, 353-356).

According to the system above, it is necessary that the RNA or the DNA used, which is transcribed to the desired RNA, whether eukaryotic or prokaryotic, be different. Various producers of proteins and the like prefer for different reasons different systems. This means that for the synthesis of a specific gene product, for instance of a certain protein, two different RNA or DNA must be produced, if the expression is desired to be possible at yields of interest for production in eukaryotic as well as in prokaryotic systems, even though the same gene sequence is used. This requirement, however, is expensive.

### SUMMARY OF THE INVENTION

It is the technical object of the invention to propose a method for cell-free gene expression in a eukaryotic system, in which an RNA can be used that can also be employed without any modification in a prokaryotic system.

### BRIEF DESCRIPTION OF THE DRAWINGS

FIG. 1 is a diagrammatic representation of the general structure of an mRNA according to an embodiment of the invention.

FIG. 2 shows the chemical structural formula of biotin ApG.

FIG. 3 is a diagrammatic representation of the structure of a pX-FA plasmid showing digestion sites for the restriction enzymes, EcoRV and P ciI.

FIG. 4 is a graphical representation of the protein yields of FABP as a function of different mRNAs coding for the fatty acid-binding protein.

FIG. 5 is a photographic representation of an eletrophoresis gel of different mRNAs prepared according to embodiments of the invention.

FIG. 6 is a photographic representation of an electgrophoresis gel showing the homogeneity of the synthesized protein prepared from different mRNAs.

FIG. 7 is a diagrammatic representation of the structure of a pIX4.0-FA-A plasmid showing the digestion sites for the restriction enzymes, BbsI and PciI.

FIG. 8 is a graphical representation of the protein yields of FABP as a function of mRNA prepared according to Example 4.

FIG. 9 is a graphical representation of the protein yields of FABP as a function of mRNA prepared according to Example 3a.

### DETAILED DESCRIPTION OF THE PREFERRED EMBODIMENTS

For achieving this technical object, the invention teaches a method of the above species, which is characterized by that the mRNA matrix, viewed in the 5′-3′ direction, comprises a Shine Dalgarno sequence, thereto preferably immediately connected to a first spacer sequence and thereto preferably immediately connected to the gene sequence.

The invention is based on the surprising finding that gene expression is possible in a prokaryotic system as well as in a eukaryotic system with an RNA, even when it does not contain a Kozak sequence, but rather the Shine Dalgarno sequence being typical for prokaryotes as an initialization sequence.

By the invention it is achieved that with a single mRNA (or a DNA coding therefor), a defined gene product can be expressed according to the choice of the user, either in a prokaryotic system or in a eukaryotic system. Thereby, the construction of different RNA or DNA for the same gene, but different systems is not required, so to speak “a universal product” for both systems is obtained. It is in principle possible to use all genes, which can be expressed in the respective system. Sequences of desired genes, as DNA or mRNA, can be taken from the publicly accessible gene data banks.

For the invention, it has been found that the mRNA for gene expression in a eukaryotic system need not necessarily comprise the eukaryote-typical elements: 5′-terminal cap, Kozak sequence and 3′-terminal poly (A) tail, if, instead, one or more of the following prokaroyte-typical elements are provided: 5′ UTR, secondary structure at the 5′ end (hairpin), Shine Dalgarno sequence and/or transcription terminator originating from bacteriophages. In particular, instead of a cap, a biotin residue may be arranged at the 5′-terminal end.

Cap represents 7-methyl GTP and occurs in nature in 3 different forms: all caps contain 7-methyl guanylate, which is linked by a triphosphate binding to the ribose at the 5′ end. Cap 0 has no methylated ribose, cap 1 has one, and in cap 2, two ribosomes are methylated at the C2 oxygen atom. The 7-Methyl-GTP is required for the localization of the mRNA at the ribosome. Poly (A) protects the 3′ end from the decomposition by exonucleases.

In a preferred embodiment, no ATG and/or AUG with open reading frame are arranged in the 5′ section of the RNA matrix in front of the Shine Dalgarno sequence.

In principle, the 5′-terminal end of the RNA may be unmodified. An improvement of the yield is achieved, however, if the 5′-terminal end of the nucleotide sequence of the RNA matrix is formed of a cap structure or carries a biotin residue.

Between the 5′-terminal end of the RNA matrix and the Shine Dalgarno sequence, an enhancer sequence, in particular a 5′-non-translated sequence (5′ UTR) originating from bacteriophages, may be arranged. This is recommended, in particular in order to increase a translation rate, in particular in prokaryotes. Surprisingly, further, the enhancer sequence does not disturb eukaryotic systems.

It may be provided that between the 5′-terminal end of the RNA matrix and the enhancer sequence, a hairpin structure is arranged. This is also effective in eukaryotic systems for protection against decomposition. Natural, efficiently translated eukaryote-typical mRNAs do not, however, have strong secondary structures at their 5′ end, since otherwise the translation initiation is inhibited because of poor accessibility of the cap to initiation factors (Hershey J W B and Merrick W C, 2000, The pathway and mechanism of initiation of protein synthesis. P. 33-88, in: Translational control of gene expression. Eds. Sonenberg N, Hershey J W B and Mathews M B, Cold Spring Harbour Laboratory Press, New York). Corresponding considerations apply, if the 3′-terminal end of the RNA matrix is formed by a transcription terminator originating from bacteriophages, in particular, by a hairpin structure.

The first spacer sequence may be formed of 3 to 20, preferably 5 to 15, nucleotides, preferably pyrimidine-rich nucleotides, but without a purine sequence, however. Between the gene sequence and the 3′-terminal end of the RNA matrix, a second spacer sequence may be arranged, which preferably has a length from 10 to 50, in particular 20 to 40, nucleotides.

In detail, the RNA matrix may comprise the following structure elements optionally immediately following each other, beginning from the 5′-terminal end: cap or biotin, optionally hairpin, optionally enhancer sequence, Shine Dalgarno sequence, first spacer sequence, gene sequence, optionally second spacer sequence, transcription terminator.

RNA, according to the invention, may, for example, be used in translation systems, which were obtained from the following cells or cell lines as lysates: BHK21 (hamster), MOLT-4 (human), MOPC 21 (mouse), RPMI 8226 (human), Jurkat FHCRC (human), HL60 (human), HEK 293 (human), CHO (hamster), HeLa (human), PC12 (rat), Sf9 (insect), Sf21 (insect), COS-1 (monkey), COS-7 (monkey), D2 (drosophila), NIH 3T3 (mouse), Tn5 B1-4 (insect), and Tn 368 (insect). Further, lysates of animal and vegetable origin can be used, such as for instance, rabbit reticulocyte lysate or wheat germ lysate. Preferred lysates are obtained from insect cells. The suitable Shine Dalgarno sequences and enhancer sequences can easily be taken from the public gene data banks of the selected insect species.

The invention further relates to a kit for gene expression in a cell-free translation system, optionally eukaryotic or prokaryotic, comprising the following components: a) transcription and/or translation system from eukaryotic cells, in particular insect cells, b) RNA matrix, which, viewed in the 5′-3′ direction, comprises a Shine Dalgarno sequence, thereto preferably immediately connected a first spacer sequence and thereto preferably immediately connected a gene sequence, or DNA coding for such an RNA. The kit may, also comprise a transcription and/or translation system from prokaryotic cells, such that a user can choose between the systems. If DNA is used, it is understood that the respective systems contain the necessary transcription factors.

The invention further relates to an mRNA comprising a sterical protecting group (not, however, a cap) at the 5′-terminal end, a Shine Dalgarno sequence, and at the 3′ end of the Shine Dalgarno sequence, a gene sequence. As a sterical protecting group, for example, one of the following structures may be used: biotin digoxigenin, fluorophores such as fluorescein, Cy3, Cy5, Bodipy, Alexa, Atto; Gp₄G; Ap₃G; G; m⁷Gp₄G; m⁷Gp₃m⁷G; m⁷Gp₄ m⁷G; benz⁷Gp₃G; benz⁷Gp₄G; benz⁷, 3′OMeGp₄G; et⁷Gp₃G; m⁷, 3′OMeGp₃G; m⁷, 3′OMeGp₄G; m⁷, 2′OMeGp₄G; m⁷Gp₅G; m⁷, 3′OmeGp₅G; m⁷, 2′deoxyGp₃G; m⁷, 2′deoxy Gp₄G; m⁷GpCH₂ppG; m⁷GppCH₂pG; Gp₃G; m⁷, 3′OMeGp CH₂ppG; m⁷, 3′OmeGppCH₂pG; m⁷,2′OMeGppCH₂pG; m⁷GpCH₂ppm⁷G.

The invention also comprises RNA hybrids with oligomers from DNA, RNA, PNA and modified forms therefrom, which may also be modified with further chemical groups, as mentioned above.

The invention finally relates to a DNA coding for an RNA according to the invention.

With respect to further features of the RNA or DNA, separately or in the kit, reference is made to the above explanations.

In the following, the invention is described with respect to embodiments representing examples of execution only.

### EXAMPLE 1

### mRNA According to the Invention

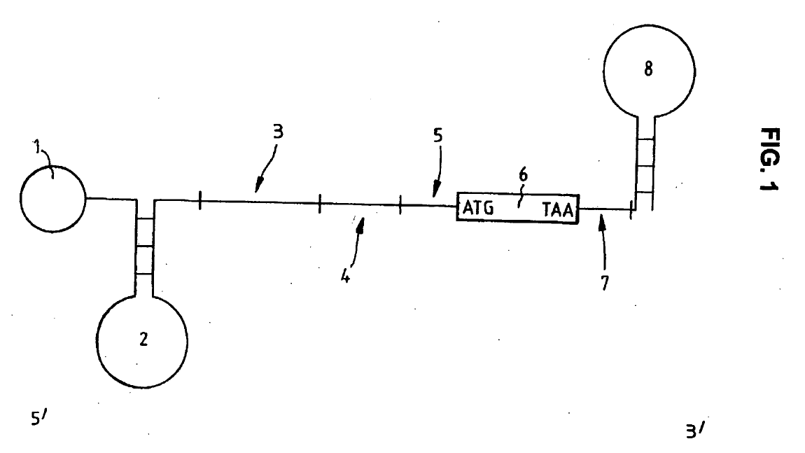

FIG. 1 shows the general structure of an mRNA according to the invention. First, a biotin residue 1 can be seen. Instead, a different sterical protecting group, if applicable also a 7-methyl guanosine group as a cap, may be provided. Then follow a hairpin structure 2 and a phage enhancer sequence 3. Then follows the Shine Dalgarno sequence 4. A first spacer sequence 5 consisting of 5 to 8 nucleotides, which are mainly pyrimidine bases, is connected to the gene sequence 6 coding for the gene to be expressed. Then follows a second spacer sequence 7 with 20 to 40 nucleotides, which may practically be arbitrary. At the 3′-terminal end, again, a hairpin structure 8 is arranged.

The selection of the Shine Dalgarno sequence takes place according to the eukaryotic cell species, which represent the basis for the translation system.

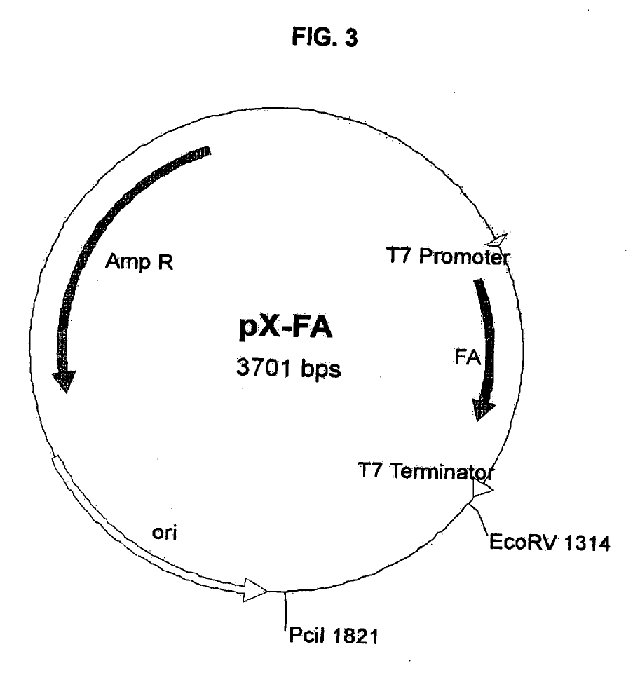

A specific sequence, which is suitable, for example, for the expression of the protein “fatty acid-binding protein from bovine heart” in an expression system on the basis of cells of the insect species, is indicated in sequence SEQ ID NO: 1 as an example only. It is transcribed from the plasmid pXFA, the vector card of which is shown in FIG. 3 and the sequence of which is shown in SEQ ID NO: 2. It is understood that any other gene sequences, Shine Dalgarno sequences etc. may be used, which have to be selected according to the desired protein and the desired expression systems only.

### Example 2

### Production of the mRNA According to Example 1

The plasmid pXFA (FIG. 3, SEQ ID NO: 2) was digested with the restriction enzymes EcoRV and PciI (NEB) according to manufacturer's instructions. The residual vector resulting herefrom (3,196 bp) was separated electrophoretically in the agarose gel from the cut-out plasmid fragment and incompletely digested plasmid. The gel part with the residual vector was cut out, therefrom the DNA was purified by means of gel elution (High Pure PCR Product Purification Kit, Roche) according to manufacturer's instructions, and the DNA concentration was determined photometrically by the absorption of light at 260 nm.

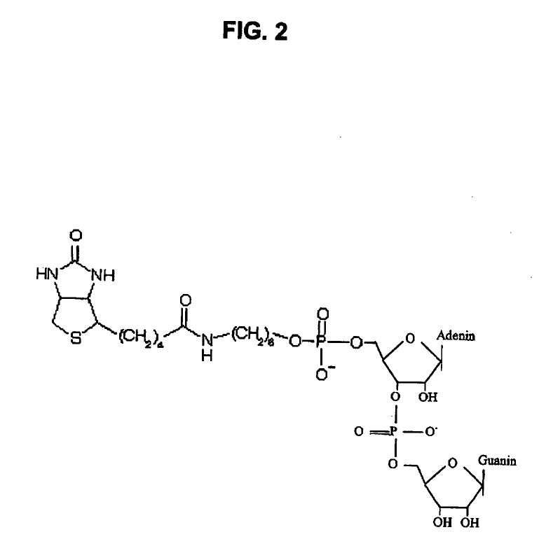

The DNA was transcribed in RNA with the EasyXpress Protein Synthesis Insect Kit (Qiagen) according to manufacturer's instructions, with one exception: instead of the NTP mix of the kit, the following components were used with the final concentrations referred to the transcription reaction: 3.75 mM each of ATP, CTP and UTP, 1.5 mM of GTP (all Roche) and 2 mM of biotin ApG (application synthesis Noxxon AG, Berlin, Germany, FIG. 2).

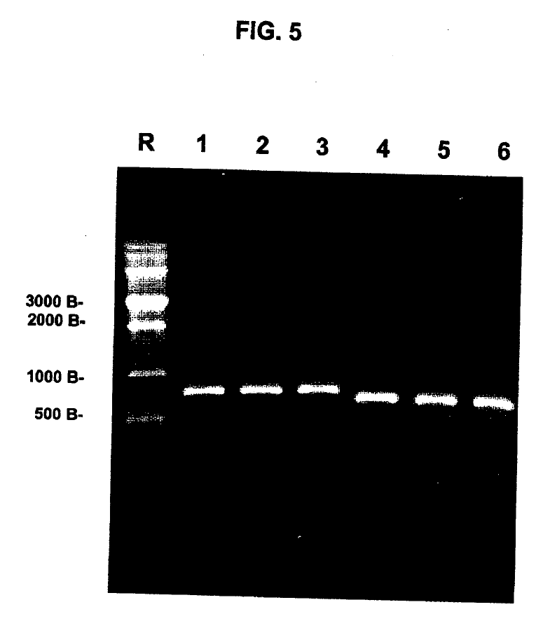

After transcription, the reaction batch was reacted with 0.5 μl 10 U/μl RNase-free DNaseI (Roche) for the elimination of the DNA and incubated for 30 min at 37° C. Then, the reaction batch was purified with the purification system of the kit. The RNA concentration of the purified transcription batch was determined photometrically by the absorption of light at 260 nm and analyzed gel-electrophoretically (FIG. 5).

Another mRNA was produced as mentioned above, with the following exception: instead of the NTP mix of the kit, the following components were used with the final concentrations referred to the transcription reaction: 3.75 mM each of ATP, CTP and UTP, 1.5 mM of GTP (all Roche) and 0.5 mM of P1,P3-di(guanosine-5′)triphosphate (Catalog No. D1012, Sigma). The mRNA was further processed as mentioned in Example 2 and analyzed gel-electrophoretically (FIG. 5).

### EXAMPLE 3

### Expression of the Proteins of Example 2 in a Eukaryotic System

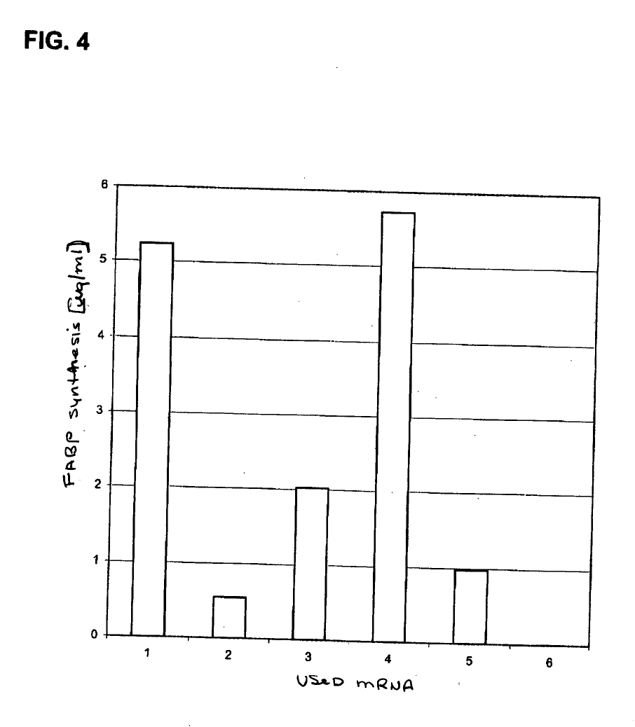

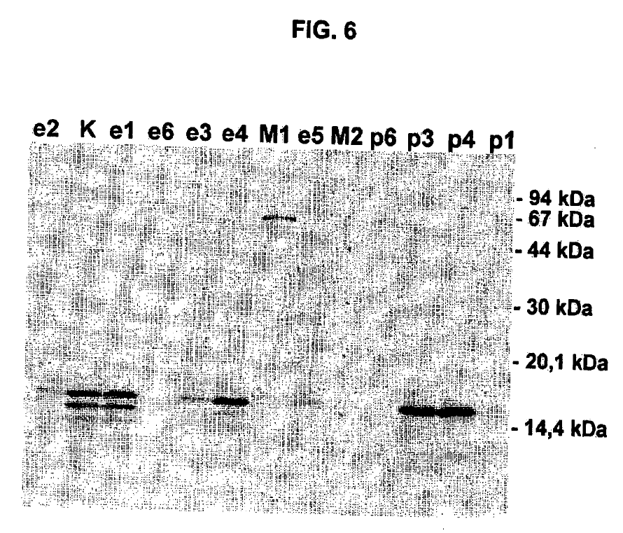

The first mRNA described in Example 2 was used, referred to the translation reaction, in a final concentration of 600 nM for the cell-free translation performed according to manufacturer's instructions with the EasyXpress Protein Synthesis Insect Kit (Catalog No. 32552, Qiagen). In addition, ¹⁴C-marked valine was used for the translation, such that a molar activity of 80 dpm/μmol has been reached. The protein yields were quantified by means of the incorporation of the radioactively marked amino acid reaction product insoluble in hot trichloro acetic acid and measurement in the scintillation counter (No. 3, FIG. 4). 2 μg of the protein per ml reaction solution were obtained. The homogeneity of the synthesized protein was analyzed gel-electrophoretically (FIG. 6).

The second RNA mentioned in the example was translated as above and the protein yield was quantified (No. 4, FIG. 4). 5.7 μg/ml reaction solution of the protein were obtained. The homogeneity of the synthesized protein was analyzed gel-electrophoretically (FIG. 6).

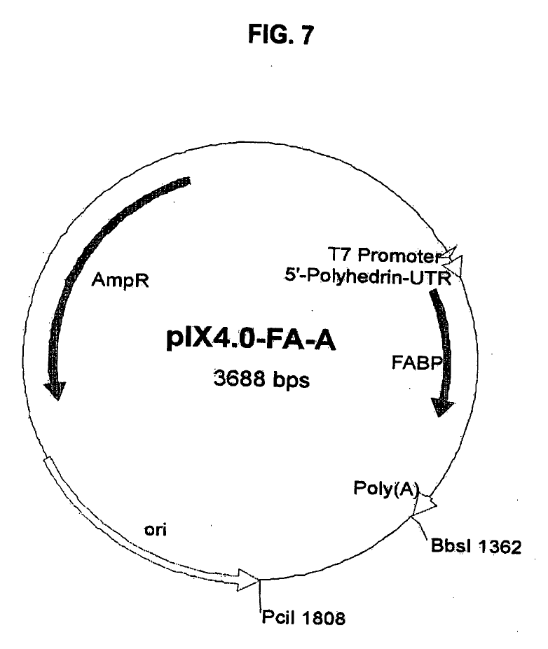

For No. 1-2, the plasmid pIX4.0-FA-A comprising eukaryotic regulation elements (FIG. 7, SEQ ID NO: 3) was digested with the restriction enzymes BbsI and PciI, purified as indicated in Example 2, and the DNA concentration was determined. The DNA was used for the transcription reaction as described in Example 2. Instead of the NTP mix of the kit, the following components were used with the final concentrations referred to in the transcription reaction: No. 1: 3.75 mM each of ATP, CTP and UTP, 1.5 mM of GTP, 0.5 mM cap (m⁷G(5′) ppp(5′)G, Catalog No. 8050, Ambion); No. 2: same components as for No. 5. The RNA nucleotide sequence resulting from the transcription of the plasmid fragment from pIX4.0-FA-A is shown in 4 SEQ ID NO: 4. All mRNAs were analyzed gel-electrophoretically (FIG. 5).

All transcription batches described here were used for the translation reaction, as described in Example 2, second part, and the respective protein yields were determined. For No. 6 RNase-free water was used instead of RNA. All protein synthesis reactions were analyzed gel-electrophoretically (FIG. 6).

### Example 3a

### Expression of the Proteins from Example 2 in a Eukaryotic System Based on Mammal Cells

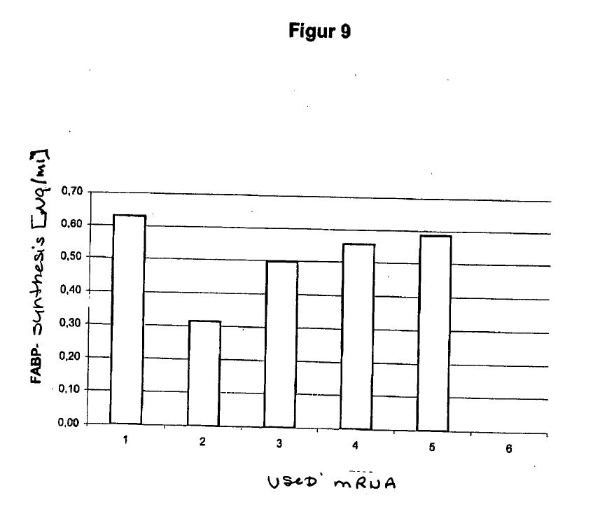

The first mRNA described in Example 2 was used, referring to the translation reaction, in a final concentration of 300 nM for the cell-free translation performed according to manufacturer's instructions with the TNT Reticulocyte Lysate System (Catalog No. L4610, Promega). In addition, ¹⁴C-marked leucine was used, such that a molar activity of 706 dpm/μmol has been reached. The protein yields were quantified by means of the incorporation of the radioactively marked amino acid reaction product insoluble in hot trichloro acetic acid and measurement in the scintillation counter (No. 3, FIG. 9). 0.5 μg of the protein per ml reaction solution were obtained.

The second mRNA mentioned in the example was translated as above and the protein yield was quantified (No. 4, FIG. 9). 0.56 μg of the protein per ml reaction solution were obtained.

### EXAMPLE 4

### Expression of the First Protein from Example 2 in a Prokaryotic System

The RNA was used, referring to the translation reaction, in a final concentration of 400 nM for the cell-free translation performed according to manufacturer's instructions with the in-vitro-PBS-Kit based on Escherichia coli cells (Catalog No. P-1102, RiNA GmbH, Berlin). In addition, ¹⁴C-marked valine was used for the translation, such that a molar activity of 3.35 dpm/μmol has been reached.

The protein yields were quantified by means of the incorporation of the radioactively marked amino acid reaction product insoluble in hot trichloro acetic acid and measurement in the scintillation counter. The homogeneity of the synthesized protein was analyzed gel-electrophoretically (FIG. 6).

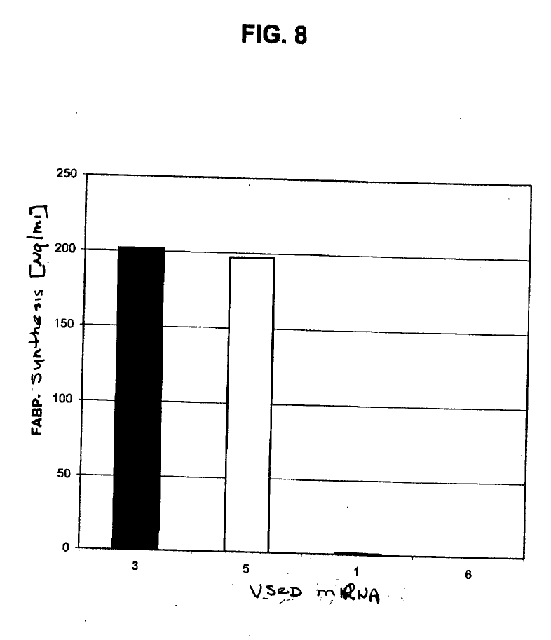

200 μg/ml of reaction solution of the protein were obtained (see No. 4, FIG. 8).

### EXAMPLE 5

### Results

FIG. 4 shows protein yields as a function of the different mRNAs coding for the fatty acid-binding protein coding. Control mRNAs were produced as described in Example 2, with the following deviations. For No. 5, instead of the NTP mix of the kit, the following components were used with the final concentrations, referred to the transcription reaction: 3.75 mM each of ATP, CTP and UTP, 1.5 mM of GTP.

FIG. 5 shows an electrophoretic analysis of the different mRNAs. Of each mRNA, 200 ng were applied on the agarose gel, and the gel was stained after the decomposition with ethidium bromide. The numbering of the traces corresponds to the numbering of the mRNAs in FIG. 4. R: RNA size standards; K: control RNA. The following mRNAs were used (track number):

No. 1: eukaryote-typical mRNA with cap, effective insect-specific 5′ UTR from baculoviral polyhedrin gene and poly (A) sequence at the 3′ end.

No. 2: mRNA as in No. 1 without cap.

No. 3: mRNA according to the invention without cap, instead with biotin at the 5′ end, without insect-specific 5′ UTR and without Kozak sequence, instead with prokaryote-typical 5′ UTR and strong secondary structure at the 5′ end, without poly (A) sequence at the 3′ end, instead with prokaryote-typical 3′ UTR with T7 page transcription terminator at the 3′ end.

No. 4: mRNA according to the invention as in No. 3 without cap, instead with P1,P3-Di (guanosine-5′)triphosphate at the 5′ end, without insect-specific 5′ UTR and without Kozak sequence, instead with prokaryote-typical 5′ UTR and strong secondary structure at the 5′ end, without 3′ poly (A), instead with prokaryote-typical 3′ UTR with T7 page transcription terminator at the 3′ end.

No. 5: mRNA as in No. 3 without biotin.

No. 6: without mRNA.

FIG. 6 shows the electrophoretic analysis of the homogeneity of the synthesized protein starting from the different mRNAs.

The protein synthesis batches described in the examples 3 and 4 were separated in the SDS polyacrylamide gel, and an autoradiogram of the gel was determined. e: expression in the eukaryotic system, p: expression in the prokaryotic system. The numbers correspond to the numbering in FIG. 4. M1: marker protein, M2: non-radioactive marker protein (invisible in the autoradiogram).

As can be seen from FIG. 4, high yields of fatty acid-binding protein are achieved in the employed eukaryotic cell-free translation system with the mRNA according to the invention with Shine-Dalgarno sequence and strong secondary structure at the 5′ end, without cap at the 5′ end, without Kozak sequence, without insect-specific 5′ UTR efficiently increasing the translation and without poly (A) sequence at the 3′ end (No. 3 and 4) with 2 or 5, 7 μg/ml, compared to the eukaryote-typical mRNA without Shine Dalgarno sequence, with cap at the 5′ end, with insect-specific 5′ UTR and poly (A) sequence at the 3′ end (No. 1).

Furthermore, the eukaryote-typical mRNA without modification at the 5′ end (No. 2) is translated with little efficiency only. This shows that the expression strength for this mRNA strongly depends on the presence of a cap. In contrast thereto, the prokaryote-typical mRNA without modification at the 5′ end (No. 5) is translated approximately two times as efficiently, the prokaryote-typical mRNA without cap, however, with biotin at the 5′ end (No. 3) approximately three times as efficiently, and the prokaryote-typical mRNA without cap, however, with P1,P3 di(guanosine-5′)triphosphate at the 5′ end (No. 4) approximately ten times as efficiently.

FIG. 6 shows that the synthesized fatty acid-binding protein (14.8 kDa) is detected with the expected molecular weight. For the eukaryote-typical mRNA, the synthesized protein is detected in the form of three bands (track e1). In contrast thereto, the protein substantially appears in the form of one band, if the mRNA according to the invention is used as a matrix for the protein synthesis in the eukaryotic and prokaryotic protein synthesis system (tracks e3, e4, p3 and p4).

FIG. 8 shows protein yields in the prokaryotic system as a function of the different mRNAs coding for the fatty acid-binding protein. For the production of the mRNA (No. 3) and of the comparison mRNAs (No. 1, 5 and 6), reference is made to FIG. 4 and the accompanying text. As can be seen from FIG. 8, the mRNA according to the invention (No. 3) is very efficiently translated with a 200 μg/ml yield of fatty acid-binding protein. The protein yield is practically the same as for standard mRNA for this system, which does not contain biotin at the 5′ end (No. 4). In contrast thereto, the eukaryote-typical mRNA with cap at the 5′ end, with insect specific 5′ UTR and poly (A) sequence at the 3′ end (No. 1) is practically not translated at all. This shows that the system is specific for mRNA with prokaryote-typical elements, and that the modification of the prokaryotic mRNA at the 5′ end has no influence on the translation efficiency.

As can be seen in FIG. 9, which shows results for Example 3a, high yields of fatty acid-binding protein are achieved in the employed eukaryotic cell-free translation system based on mammal cells with mRNA according to the invention with Shine Dalgarno sequence and strong secondary structure at the 5′ end, without cap at the 5′ end, without Kozak sequence, without mammal cell-specific 5′ UTR efficiently increasing the translation and without poly (A) sequence at the 3′ end (No. 3, 4 and 5) with 0.50, 0.56 or 0.59 μg/ml, compared to the eukaryote-typical mRNA (No. 1).

Furthermore, the eukaryote-typical mRNA without modification at the 5′ end (No. 2) is translated with a small efficiency only. This shows that the expression strength for this mRNA strongly depends from the presence of cap. In contrast thereto, the prokaryote-typical mRNAs according to the invention
a) without modification at the 5′ end (No. 5),
b) without cap, however with biotin at the 5′ end (No. 3) and/or
c) without cap, however with P1,P3-Di(guanosine-5′) triphosphate at the 5′ end (No. 4)
are translated approx. two times as efficiently as the eukaryote-typical mRNA without modification at the 5′ end.

1. A method for gene expression in a cell-free translation system, wherein the reaction solution comprises an RNA matrix with a gene sequence, which codes for an expression product to be expressed, and a translation system from eukaryotic cells, wherein the reaction solution is incubated, and wherein the expression product is separated from the reaction solution, wherein the RNA matrix, viewed in the 5′-3′ direction, comprises a Shine Dalgarno sequence, connected to a first spacer sequence and which is connected to the gene sequence.

2. The method according to claim 1, wherein in the 5′ section of the RNA matrix, no ATG or AUG with open reading frame are arranged in front of the Shine Dalgarno sequence.

3. The method according to claim 1 wherein the 5′-terminal end of the nucleotide sequence of the RNA matrix comprises a cap or a sterical protecting group comprising a biotin residue.

4. The method according to claim 1, wherein between the 5′-terminal end of the RNA matrix and the Shine Dalgarno sequence, an enhancer sequence, comprising a 5′-non-translated sequence (5′ UTR) originating from bacteriophages, is arranged.

5. The method according to claim 1, wherein between the 5′-terminal end of the RNA matrix and the enhancer sequence, a hairpin structure is arranged.

6. The method according to claim 1, wherein the 3′-terminal end of the RNA matrix is formed by a transcription terminator originating from bacteriophages comprising a hairpin structure.

7. The method according to claim 1, wherein the first spacer sequence is formed of 3 to 10 nucleotides comprising pyrimidine-rich nucleotides.

8. The method according to claim 1, wherein between the gene sequence and the 3′-terminal end of the RNA matrix, a second spacer sequence is arranged having a length of 10 to 50 nucleotides.

9. A method according to claim 1, wherein the RNA matrix comprises the following structure elements beginning from the 5′-terminal end: biotin, a hairpin structure, an enhancer sequence, a Shine Dalgarno sequence, a first spacer sequence, a gene sequence, a second spacer sequence, or a transcription terminator.

10. The method according to claim 1, wherein the translation system was obtained from insect cells.

11. A kit for preparing gene expression in a cell-free translation system according to the method of claim 1, comprising the following components:
a) translation system from eukaryotic cells, in particular insect cells,
b) RNA matrix, which, viewed in the 5′-3′ direction, comprises a Shine Dalgarno sequence connected to a first spacer sequence which is connected to a gene sequence or DNA vector coding for such an RNA.

12. The kit according to claim 11, wherein in the 5′ section of the RNA matrix, no ATG or AUG with open reading frame are arranged in front of the Shine Dalgarno sequence.

13. The kit according to claim 11, wherein the 5′-terminal end of the nucleotide sequence of the RNA matrix comprises a cap or a sterical protecting group comprising a biotin residue.

14. The kit according to claim 11, wherein between the 5′-terminal end of the RNA matrix and the Shine Dalgarno sequence, an enhancer sequence comprising a 5′-non-translated sequence (5′ UTR) originating from bacteriophages, is arranged.

15. The kit according to claim 11, wherein between the 5′-terminal end of the RNA matrix and the enhancer sequence, a hairpin structure is arranged.

16. The kit according to claim 11, wherein the 3′-terminal end of the RNA matrix is formed by a transcription terminator originating from bacteriophages comprising a hairpin structure.

17. The kit according to claim 11, wherein the first spacer sequence is formed from 3 to 10 nucleotides comprising pyrimidine-rich nucleotides.

18. The kit according to claim 11, wherein between the gene sequence and the 3′-terminal end of the RNA matrix, a second spacer sequence is arranged having a length of 10 to 50 nucleotides.

19. The kit according to claim 11, wherein the RNA matrix comprises the following structure elements beginning from the 5′-terminal end: biotin, a hairpin structure, an enhancer sequence, a Shine Dalgarno sequence, a first spacer sequence, a gene sequence, a second spacer sequence, or a transcription terminator.

20. The kit according to claim 11, further comprising as component c), one or more substances from the group comprising “amino acids and metabolism components supplying energy and being necessary for the synthesis of the expression product”.

21. An RNA comprising a sterical protecting group at the 5′-terminal end that is not a cap, a Shine Dalgarno sequence and a gene sequence at the 3′ end of the Shine Dalgarno sequence.

22. The RNA according to claim 21 with the following structure elements immediately following each other, beginning from the 5′-terminal end: biotin, a hairpin structure; an enhancer sequence, a Shine Dalgarno sequence, a first spacer sequence, a gene sequence, a second spacer sequence, and a transcription terminator.

23. A DNA coding for the RNA according to claim 21.
